(19) United States
(12) Patent Application Publication
(10) Pub. No.: US 2020/0345382 A1
(43) Pub. Date: Nov. 5, 2020

(54) INLINE CUTTER FOR CUTTING AND RETRIEVING IMPLANTED MICROSURGICAL DEVICES

(71) Applicants: Bibianna Cha, Seattle, WA (US); Kurt Faulhaber, Kent, WA (US); David Scott, Bothell, WA (US)

(72) Inventors: Bibianna Cha, Seattle, WA (US); Kurt Faulhaber, Kent, WA (US); David Scott, Bothell, WA (US)

(21) Appl. No.: 16/864,096

(22) Filed: Apr. 30, 2020

Related U.S. Application Data
(60) Provisional application No. 62/841,567, filed on May 1, 2019.

(51) Int. Cl. A61B 17/295; A61B 17/30; A61B 17/29

(52) U.S. Cl. CPC A61B 17/295; A61B 2017/00345; A61B 17/2909; A61B 17/30

(57) ABSTRACT

Disclosed are systems, devices, and methods that allow a surgeon to both cut an implanted microsurgical device and also to simultaneously retain the cut-off piece using a single tool and a single motion. In various embodiments, this is achieved by a microsurgical cutting and grasping instrument that may include a piston assembly including a piston, a cap, an outer shell housing the piston and a tension member in operative engagement with the piston and the cap, a cannula connected to the piston, a rod axially disposed in the cannula which is moved in an axial direction by the piston when the outer shell and the piston are squeezed together, a cutter assembly disposed at a distal end of the rod and configured to perform both an inline cutting action and a grasping action on a target.

### CROSS-REFERENCE TO RELATED APPLICATIONS

This application claims priority to U.S. Provisional Patent Application No. 62/841,567, filed May 1, 2019, the disclosure of which is hereby incorporated by reference in its entirety.

### FIELD

The subject matter described herein generally relates to microsurgical instruments, and more particularly to instruments especially but not exclusively for cutting and retracting a microsurgical device implanted inside a body such as the Minimally Invasive Glaucoma Surgery (MIGS) devices that have been implanted inside an eye by an ophthalmic procedure.

### BACKGROUND

Currently, medical devices are oftentimes surgically introduced into patients' bodies (e.g., eyes) to treat or diagnose various symptoms. For example, very small, intraocularly implantable devices collectively referred to as Minimally Invasive Glaucoma Surgery (MIGS) devices are widely used to treat glaucoma. Presently, at least four MIGS devices are approved for use by the United States FDA. The first is the iStent Inject®, which is a pair of multi-directional stents manufactured by Glaukos that are placed in the ocular trabecular meshwork. The second is the CyPass® Micro-Stent, which is manufactured by Alcon and is placed in the ocular supra-choroidalspace to treat open-angle glaucoma during cataract surgery. The third is the iTrack™ microcatheter, manufactured by Ellex, which is inserted into Schlemm's canal to dilate an eye's natural drainage system. The fourth is the XEN® Gel Stent, manufactured by Allergan, that helps to create a filtration pathway from the anterior chamber, through the sclera, and into the subconjunctival space. Each of these devices is designed to improve aqueous fluid out-flow and to reduce intraocular pressure. These devices are surgically implanted in an area within the eye called “the angle.”

A post-approval study required by the FDA have revealed a risk of eye damage in people who have some of the devices implanted. Of the known MIGS devices, FDA has found that people who have the CyPass® devices implanted are at a risk of losing endothelial cells that line the inner surface of cornea. Endothelial cell loss is known to be associated with potential damage to the cornea including swelling, cloudiness, eye pain, reduction in vision, and the potential need for corneal implant. As a result of these post-approval study findings, Alcon announced a voluntary market withdrawal of the device and is asking physicians to stop implantation immediately. For patients who have already received the device, the manufacturer may recommend a revision surgery such as repositioning or trimming the implanted micro stent.

Surgeons currently use a multiple number of various microsurgical tools to perform revision surgery procedures related to implanted micro stents. One example is 25 g Ahmed Stent Forceps by MicroSurgical Technology (Redmond, Wash.), which may be used to grasp the stent and adjust stent position, but not to cut it. Another example is 23 g Hoffman/Ahmed Horizontal Curved Scissors by the same company, which may be used to cut the stent, but not to grasp it. Yet another example is 23 g Hoffman/Ahmed Horizontal Straight Scissors by MicroSurgical Technology, which may be used to cut the stent, but not to grasp it. However, each of these known devices lacks the specific features and does not provide the benefits of the embodiments described herein. In particular, none of the conventional surgical tools are able to both trim off the micro stent and also to retain the piece of micro stent that has been trimmed off, using a single tool. It is important to note that for the revision surgery procedures in particular, oftentimes a goniolens must also be used (manipulated by the surgeon's hand) at the same time, to have visibility into the trabecular meshwork to visualize the MIGS device. This adds an additional challenge of having only a single remaining hand to be able to hold a surgical device and manipulate it to cut and grasp a stent during a revision surgery, since the other hand must hold a goniolens at all times during the procedure.

Moreover, conventional scissors fail to trim off the micro stent at a desired cut-off angle in a single stroke, and they also fail to retain the trimmed off piece of the micro stent after trimming. Instead, a separate grasper such as the aforementioned forceps must be used to retrieve the trimmed-off piece, which oftentimes are simply dropped inside the body (e.g., in the anterior chamber of the eye) after trimming for later retrieval by a separate grasper tool, which not only adds an extra step to the revision surgery procedure for the surgeons, but more importantly possible risk of complications due to increased surgery time, increased possibility of tissue or other bodily damage to patients (e.g., due to a necessity for multiple cutting motions and use of multiple tools which must be separately inserted into the body and separately manipulated), and overall cost of operations.

It is therefore desirable to provide an improved microsurgical instrument which overcome these shortcomings.

### SUMMARY

Disclosed are systems, devices, and methods that allow a surgeon to both cut an implanted microsurgical device and also to simultaneously retain the cut-off piece using a single tool and a single motion. In various embodiments, this is achieved by a microsurgical cutting and grasping instrument that may include an actuating assembly including a piston, a cap, and a tension member in operative engagement with the piston and the cap; an outer shell housing the piston; a cannula connected to the piston; a rod axially disposed in the cannula and secured in the outer shell against axial movement; wherein the cannula is moved in an axial direction by the piston when the outer shell and the piston are squeezed together; a cutter assembly disposed at a distal end of the rod and configured to perform both an inline cutting action and a grasping action on a target.

In some embodiments, the cannula of the microsurgical cutting and grasping instrument may be moved in an axial direction by the piston when the piston is pushed toward the cap.

In some embodiments, the microsurgical cutting and grasping instrument may further include a cutter assembly disposed at a distal end of the rod and configured to perform both an inline cutting action and a grasping action on a target, the cutter assembly comprising a first cutting member and a second cutting member separated by a slot, the first cutting member provided with a first cutting edge at the first distal end and the second cutting member provided with a second cutting edge at a second distal end, each of the first and the second cutting edges having one of an arcuate or straight configuration.

In some embodiments, the first cutting edge and the second cutting edge may oppose each other such that when the cannula is moved in the axial direction towards the first and second distal ends of the cutter assembly, the first and second cutting edges may engage each other to establish the inline cutting action.

In some embodiments, the outer shell may be configured to releasably attach to a handle.

Other features and advantages of the present invention are or will become apparent to one skilled in the art upon examination of the following figures and detailed description, which illustrate, by way of examples, the principles of the present invention.

The systems, devices, and methods described herein in detail for microsurgical cutting and grasping instrument are example embodiments and should not be considered limiting. Other configurations, methods, features and advantages of the subject matter described herein will be or will become apparent to one with skill in the art upon examination of the following figures and detailed description. It is intended that all such additional configurations, methods, features and advantages be included within this description, be within the scope of the subject matter described herein and be protected by the accompanying claims. In no way should the features of the example embodiments be construed as limiting the appended claims, absent express recitation of those features in the claims.

### BRIEF DESCRIPTION OF THE DRAWINGS

Illustrated in the accompanying drawing(s) is at least one of the best mode embodiments of the present invention.

FIG. 1 shows some exemplary components of a microsurgical cutting and grasping instrument, according to some embodiments of the present invention.

FIG. 2 shows a front view of an example embodiment of the microsurgical cutting and grasping instrument, viewed from a cross section A-A (FIG. 3), according to some embodiments of the present invention.

FIG. 3 shows a side view of an example embodiment of the microsurgical cutting and grasping instrument, in a first configuration, according to some embodiments of the present invention.

FIG. 4 shows a side view of an example embodiment of the microsurgical cutting and grasping instrument, in a second configuration, according to some embodiments of the present invention.

FIG. 5 shows a side view of a cannula operatively connected to the piston in an example embodiment of the microsurgical cutting and grasping instrument, according to some embodiments of the present invention.

FIG. 6 shows a close-up view B (FIG. 3) of a cutter assembly in an example embodiment of the microsurgical cutting and grasping instrument, according to some embodiments of the present invention.

FIG. 7 shows a side view of the rod and the cutter assembly in an example embodiment of the microsurgical cutting and grasping instrument, according to some embodiments of the present invention.

FIGS. 8A-8B show a cutter assembly in a closed configuration in an example embodiment of the microsurgical cutting and grasping instrument, according to some embodiments of the present invention.

FIGS. 9A-9B show a cutter assembly in an open configuration in an example embodiment of the microsurgical cutting and grasping instrument, according to some embodiments of the present invention.

FIGS. 10A-10B show a target and a cutter assembly in an open configuration in an example embodiment of the microsurgical cutting and grasping instrument, according to some embodiments of the present invention.

FIGS. 11A-11B show a target cut and grasped by a cutter assembly in a closed configuration in an example embodiment of the microsurgical cutting and grasping instrument, according to some embodiments of the present invention.

FIGS. 12A-12B show a target freely retained by a cutter assembly in an open configuration in an example embodiment of the microsurgical cutting and grasping instrument, according to some embodiments of the present invention.

FIGS. 13A-13C show a sequence of the inline cutting action on a target by an example embodiment of the microsurgical cutting and grasping instrument, according to some embodiments of the present invention.

FIGS. 14A-14D show a sequence of the inline cutting action and the grasping action on a target by an example embodiment of the microsurgical cutting and grasping instrument, according to some embodiments of the present invention.

FIGS. 15A-15B show a side view of an example embodiment of the microsurgical cutting and grasping instrument including a retaining feature, according to some embodiments of the present invention.

FIGS. 16A-16B show a side view of another example embodiment of the microsurgical cutting and grasping instrument including another retaining feature, according to some embodiments of the present invention.

### DETAILED DESCRIPTION

Before the present subject matter is described in detail, it is to be understood that this disclosure is not limited to the particular embodiments described, as such may vary. It should also be understood that the terminology used herein is for the purpose of describing particular embodiments only, and is not intended to be limiting, since the scope of the present disclosure will be limited only by the appended claims.

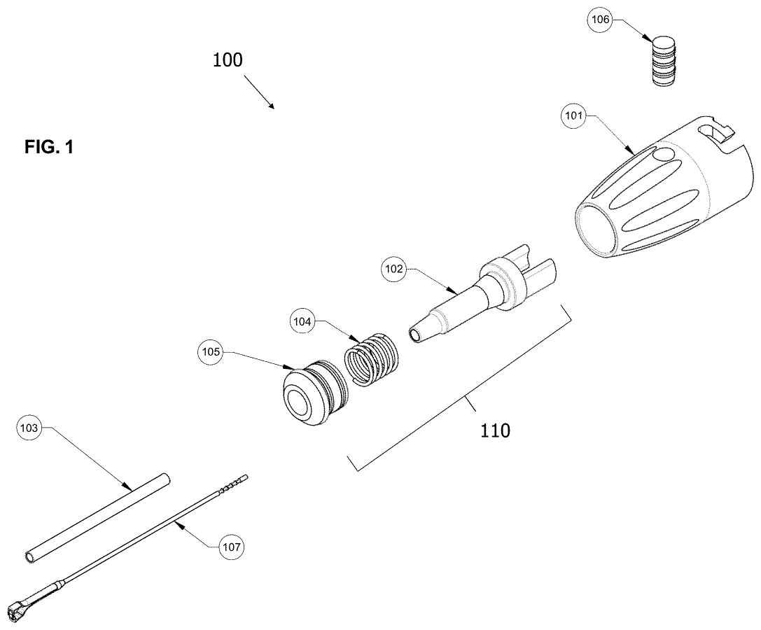

FIG. 1 shows some exemplary components of an example embodiment of the microsurgical cutting and grasping instrument 100 of the present disclosure in a disassembled state:
101—outer shell
102—piston
103—cannula
104—tension spring
105—cap
106—fastener (e.g., screw, etc.) and
107—rod, with a cutter assembly disposed on its distal end.

In some embodiments, the piston 102, tension spring 104, and cap 105 may form a actuating assembly 110, which may be encased in the outer shell 101. The piston 102 may extend axially outward from a distal end of the outer shell 101, but may be prevented from extending entirely by the tension spring 104 abutting the piston 102 and the cap 105. The cap 105 may be secured to the proximal end of the outer shell 101, e.g., by a screwing thread. Other securing means may be possible (e.g., glue, friction, pin and slot, etc.). The piston 102 may also be prevented from sliding out of the proximal end of the outer shell 101 by a fastener 106, which may be inserted laterally into the outer shell 101 to block the piston 102 from moving in the axial direction toward the proximal end of the outer shell 101.

In some embodiments, the rod 107 may be axially placed inside the cannula 103. The distal end of the rod 107 may be inserted through the cap 105 and the piston 102 of the actuating assembly 110, and further extend out into the outer shell 101 from the proximal end of the piston 102. Rod 107 may be axially secured in place by the fastener 106 that, when secured into position, e.g., screwed down, exerts a lateral force against the rod 107, when the fastener 106 is screwed onto the proximal end of the rod 107. The proximal end of rod 107 may have one or more undulations that are designed to prevent the rod 107 from moving axially when the fastener 106 is screwed against the rod 107. Other means of fastening besides screwing may be possible.

The microsurgical cutting and grasping instrument of the present disclosure may be manufactured and/or configured as a single-use device or durable and reusable device. The component materials may vary depend on the manufactured or configured type, for example, plastic for single-use and metal for durable and reusable type.

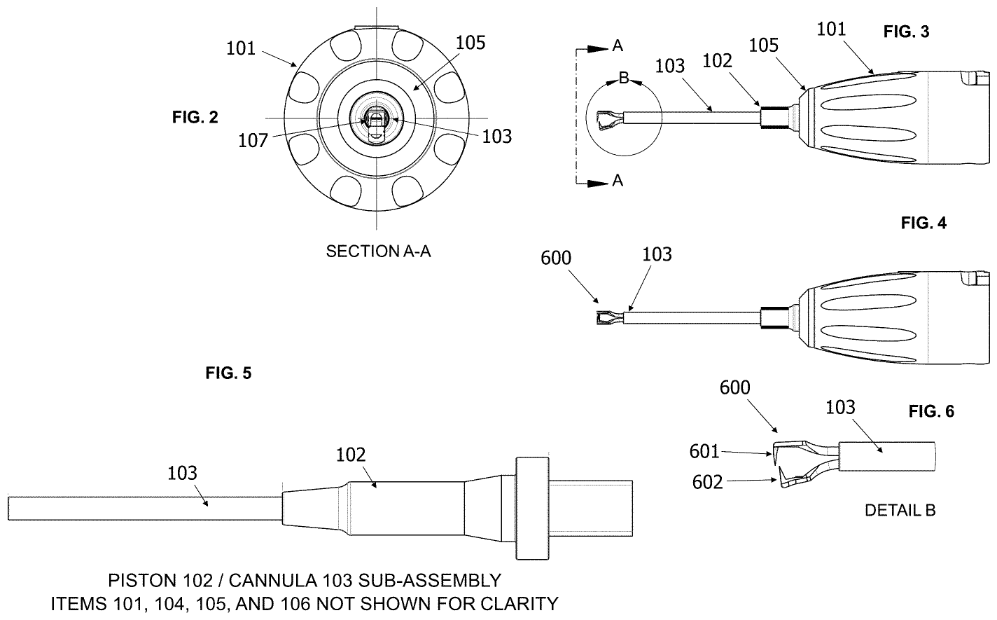

FIG. 2 shows a front view of an example embodiment of the microsurgical cutting and grasping instrument 100, viewed from the cross-section A-A (FIG. 3). As used herein, a “diameter” refers to an inner diameter, unless noted otherwise. The diameter of the cannula 103 is smaller than the diameter of the cap 105 or the diameter of the aperture on the distal end of the outer shell 101, as well as the largest lateral dimension of the cutting assembly, but the diameter of the cannula 103 is larger than the largest diameter of the rod 107.

FIG. 3 shows a side view of an example embodiment of the microsurgical cutting and grasping instrument 100, in a first configuration. In the first configuration, the cannula 103 is not pushed (or pushed enough) axially toward the distal ends of the cutting/cutter assembly 600 (FIG. 6) along the two cutting members 601 and 602, and the cutter assembly 600 is open.

FIG. 4 shows a side view of an example embodiment of the microsurgical cutting and grasping instrument 100, in a second configuration. In the second configuration, the cannula 103 is pushed axially toward the distal ends of the cutting assembly 600 (FIG. 6) along the two cutting members 601 and 602, causing the two opposing cutting edges to engage each other; the cutter assembly 600 is closed.

FIG. 5 shows a side view of a cannula 103 operatively connected to the piston 102 in an example embodiment of the microsurgical cutting and grasping instrument 100. The diameter of the cannula 103 is smaller than the aperture on the distal end of the piston 102. The combined axial length of the cannula 103 and the piston 102 (except the length of the piston 102 that extends beyond the fastener 106 when placed inside the outer shell 101) is less than the total axial length of the rod 107 and the cutter assembly 600 combined.

FIG. 6 shows a close-up view B (FIG. 3) of a cutter assembly 600 in an example embodiment of the microsurgical cutting and grasping instrument 100. The cutter assembly 600 may include a first cutting member 601 and a second member 602 which are separated by a slot. One or both of the first cutting member 601 and the second cutting member 602 may be bent, such that the first cutting edge and/or the second cutting edge is vertically offset from the major lateral plane of the rod 107.

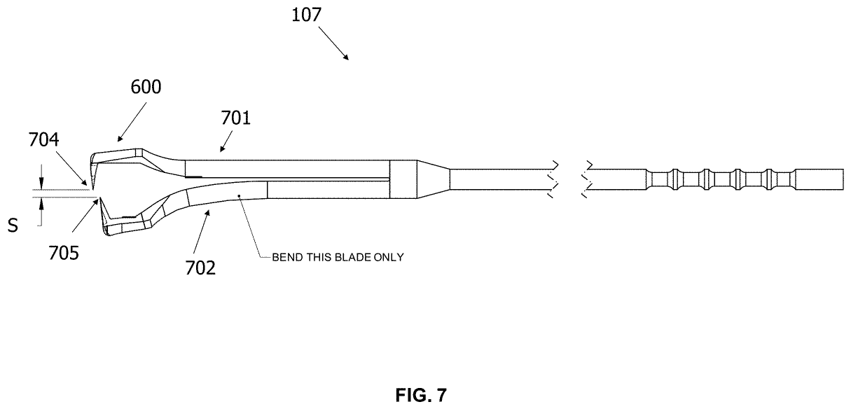

FIG. 7 shows a side view of the rod 107 and the cutter assembly 600 in an example embodiment of the microsurgical cutting and grasping instrument 100. In this example, a first cutting member (blade) 702 is bent, while the second cutting member 701 is not bent. The first cutting edge 704 and the second cutting edge 705 are spaced apart when the cutter assembly 600 is in an open configuration. For example, the space S between the first cutting edge and the second cutting edge may be about 0.006 inches (or 0.1524 mm) to about 0.020 inches (or 0.508 mm) for performing stent revision surgeries, but other value may be possible depending on the particular needs of the surgeon and the target of the surgical procedure performed by the surgeons. In some embodiments, only one cutting member (blade) (e.g., cutting member 702) is bent, but in some embodiments, both the first and the second cutting member may be bent. In either case, the bend should be of a degree such that the two opposing cutting edges 704 and 705 align or engage correctly to perform a cutting action on a target. Also, the two cutting edges provide a guide for preventing the target from undesirably moving, e.g., rolling, during the cutting action. The cutter assembly, including the cutting members and the cutting edges, may be made of a surgical grade material, such as heat-treated stainless steel, titanium, etc. In some embodiments, only one cutting edge is curved out, similar to a guillotine. In another embodiment, both the first and the second cutting edges may be curved out, or both straight. The cutting action by the first cutting edge and the second cutting edge may be similar to a scissor action, such that the cutting edges may vertically cleave the target without requiring a lot of force for the action.

In some embodiments, the rod 107 may be manufactured as a single piece. In some embodiments, the rod 107 may be manufactured as a single piece with different distal component members, for example, where the distal component members are different from cutting members 701 and 702. The different component members may have different purposes and functions. In some embodiments, the rod 107 may comprise of multiple component members manufactured in multiple pieces, for example where the cutting members 701 and 702 may be manufactured separately and attachable together. In these latter embodiments, different component members for different purposes and functions (e.g., different from cutting members 701 and 702) may be attached to the rod 107.

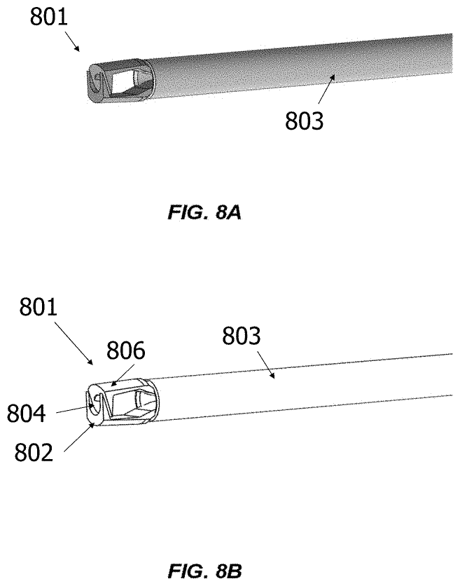

FIGS. 8A-8B show a cutting assembly 801 in a closed configuration in an example embodiment of the microsurgical cutting and grasping instrument of the present disclosure. In some embodiments, the cannula 803 may be fully pushed axially against the cutter assembly 801 to a point where the lateral dimension of the cutting assembly 801 is greater than the diameter of the cannula 803. In some embodiments, the closed configuration may be achieved even if the cannula 803 is not fully pushed axially against the cutter assembly to that same point. In some embodiments, only the first cutting edge 802 is not straight. For example, the first cutting edge 802 may have a curved-out portion 804. The curved-out portion 804 may be shaped as a slot and can be any size to accommodate a target (foreign body such as the intraocular implantable devices) the microsurgical cutting and grasping instrument 800 is designed to cut—for example, it may be 0.020″ or 0.508 mm. For example, the curved-out portion 804 may be shaped as an arc, semi-circle, U-shape, V-shape, W-shape, etc. In some embodiments, both the first cutting edge 802 and the second cutting edge 806 may be straight, or both may be non-straight.

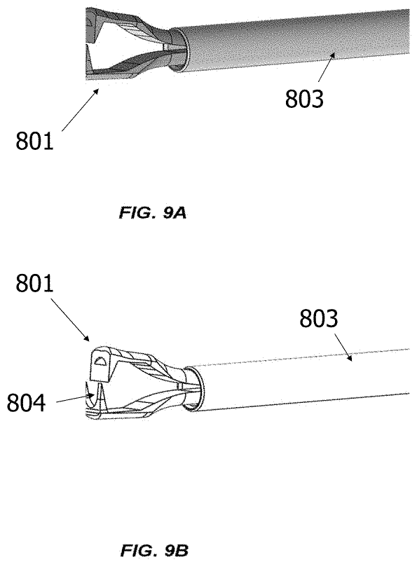

FIGS. 9A-9B show a cutting assembly 801 in an open configuration in an example embodiment of the microsurgical cutting and grasping instrument of the present disclosure. The cannula 803 is not pushed (or pushed fully) axially against the cutter assembly 801, such that the first cutting edge and the second cutting edge has a gap therebetween. In some embodiments, the first cutting edge has a curved-out portion 804, such that a target may sufficient rest therein. For example, in some embodiments, at least 50% of the diameter/lateral dimension of the target is subsumed inside the curved-out portion 804. In some embodiments, up to all of the target lays in the curved-out portion 804. When the target sufficiently rests inside the curved-out portion 804, a sufficient friction is applied to the target, such that the target is prevented from rolling when the cutting action is applied to the target.

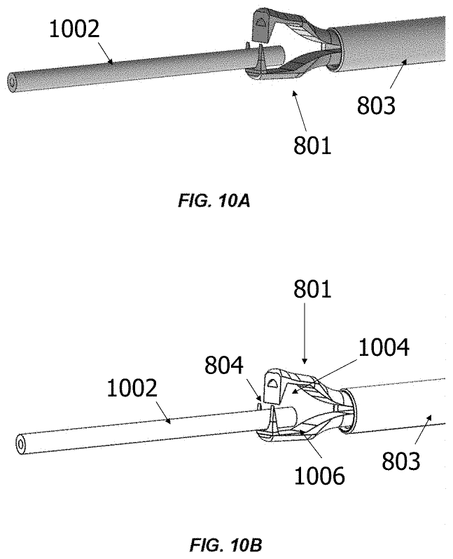

FIGS. 10A-10B show a target 1002 and a cutting assembly 801 in an open configuration in an example embodiment of the microsurgical cutting and grasping instrument of the present disclosure. In some embodiments, the width or height of the curved-out portion 804 is greater than the lateral dimension (outer diameter) of the target. In some embodiments, the width or height of the curved-out portion 804 may be equal to or less than the lateral dimension (outer diameter) of the target.

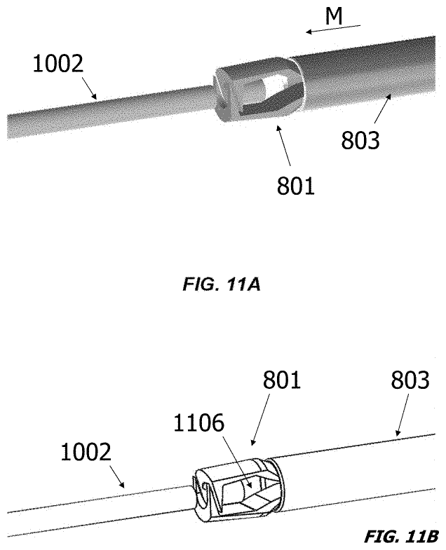

FIGS. 11A-11B show a target 1002 cut by an inline cutting action and grasped by a cutter assembly 801 in a closed configuration in an example embodiment of the microsurgical cutting and grasping instrument of the present disclosure. In some embodiments, the first cutting edge and the second cutting edge form a substantially vertically straight cut on the target 1002, such that the two cut surfaces of the target 1002 have a substantially flat lateral plane, i.e., the inline cutting action forms a substantially straight cross-section on the target. In some embodiments, the cut or trimmed off end of the target 1002 may be sufficiently grasped by the cutter assembly 801. For example, the first distal end and the second distal end of the cutter assembly 801 are each configured with a recess opposing one another and are movable relative to one another, such that when the cannula 803 is moved in the axial direction towards the first and second distal ends of the cutter assembly 801, the two opposing recesses 1004, 1006 (FIG. 10B) establish the grasping action. In some embodiments, the grasping action includes forming the two opposing recesses together into a common recess 1106 for retaining the target 1002 after the inline cutting action. In some embodiments, the inline cutting action and the grasping action are established with a single movement of the cannula 803 in the axial direction, as can be seen from the progression of the axial movement M of the cannula 803 toward the distal ends of the cutter assembly 801 as shown in FIGS. 10A-10B to 11A-11B.

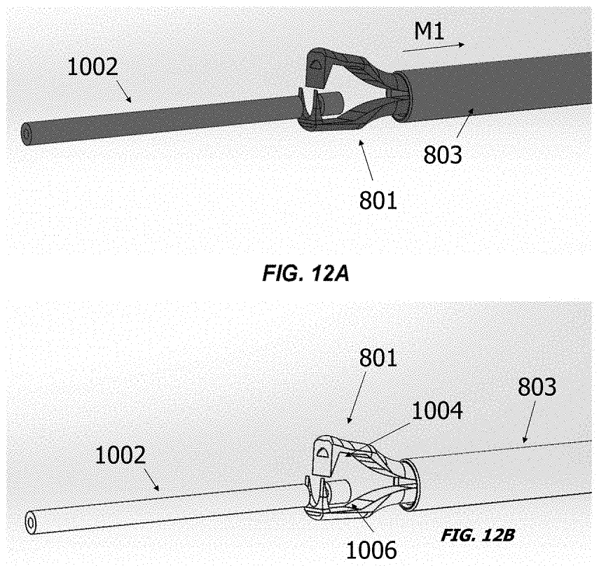

FIGS. 12A-12B show a target 1002 freely retained by a cutting assembly 801 in an open configuration in an example embodiment of the microsurgical cutting and grasping instrument of the present disclosure, subsequent to the inline cutting action in FIGS. 11A-11B. In some embodiments, one or both of the first distal end and the second distal end of the cutter assembly 801 are configured with a recess 1004/1006, such that when the cannula 803 is moved in the axial direction M1 away from the first and second distal ends of the cutter assembly 801, the target remains in the recess.

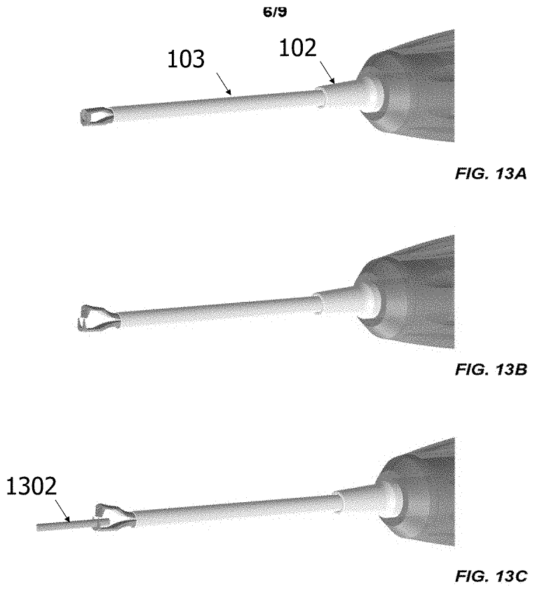

FIGS. 13A-13C show a perspective view of a sequence of the inline cutting action on a target by an example embodiment of the microsurgical cutting and grasping instrument of the present disclosure. FIG. 13A shows the cutter assembly in a closed configuration. The closed configuration may be achieved by a force/pressure exerted against the piston 102, which in turn causes the cannula 103 to axially move toward the distal ends of the cutter assembly. For example, in some embodiments, the microsurgical cutting and grasping instrument may be releasably attached to a surgical handle, which is configured to exert pushing force against the piston 102 when activated. For example, in some embodiments, the handle may be MST 360 Handle manufactured by Microsurgical Technology. The pushing force is exerted against the piston when a surgeon laterally squeezes the handle. The handle may be equipped with two opposing blades toward the distal end, which are each connected to a lever arm, both of which are in turn connected to a cylindrical piece that are housed in the distal end of the handle. By squeezing together the two blades, the cylindrical piece would protrude further outward. When a head assembly such as the microsurgical cutting and grasping instrument of the present disclosure is attached to the handle, such squeezing would cause the cylindrical piece to push against the piston 102, which in turn causes the piston 102 to axially move toward the cap 105, which causes the cannula 103 to move in the axial direction as well, causing the cutter assembly to close. FIG. 13B shows the cutter assembly in an open configuration. For example, the open configuration may be achieved when the surgeon reduces or releases pressure on the handle (the two blades). For example, the tension spring 104 causes the piston 102 to axially move away from the cap 105, such that the cannula 103 axially moves away from the distal ends of the cutter assembly. FIG. 13C shows the same configuration as FIG. 13B, except the target 1302 is now acquired inside the cutter assembly.

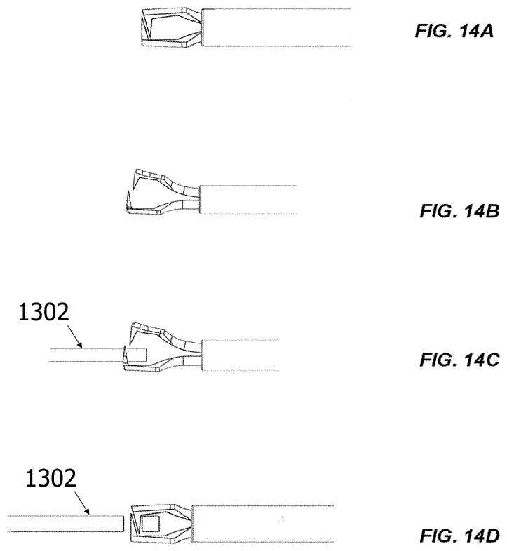

FIGS. 14A-14D show a side view of a sequence of the inline cutting action and the grasping action on a target by an example embodiment of the microsurgical cutting and grasping instrument of the present disclosure, similar to FIGS. 13A-13C above (except FIG. 14D). FIGS. 14A-14C are equivalent to FIGS. 13A-13C, respectively. FIG. 14D shows the incline cutting action and the grasping action performed on the target.

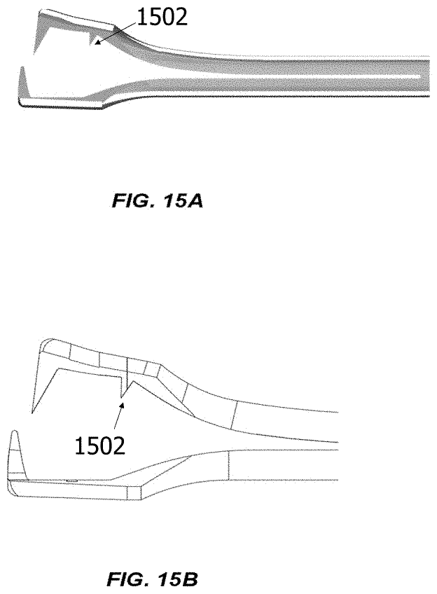

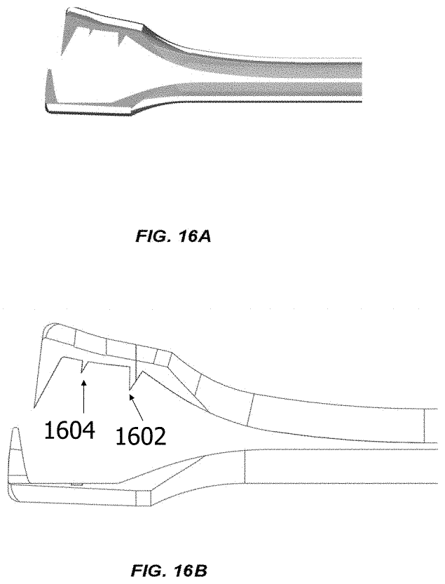

FIGS. 15A-15B show a side view of some embodiments of the microsurgical cutting and grasping instrument of the present disclosure, including a retaining feature. In some embodiments, for example, the retaining feature is a tooth member 1502 protruding from either the first cutting member or the second cutting member into the respective open recess, and into the common recess when the cutter assembly is closed during the inline cutting action or the grasping action. The retaining feature is configured to retain the target (at least the potion of the target to be cut or trimmed off) in one or both of the open recesses or the common recess prior to, during, and/or after the target being cut by an inline cutting action, and/or during the grasping action. In FIGS. 15A-15B, the tooth member 1502 is positioned on the second cutting member. The number of the retaining feature may be more than one. For example, FIGS. 16A-16B show a side view of another example embodiment of the microsurgical cutting and grasping instrument of the present disclosure, including two teeth members 1602, 1604. The size and location of each of the retaining features may vary. For example, FIGS. 16A-16B show a first tooth member and a second tooth member protruding from the second cutting member, where the first tooth member is smaller than the second tooth member, and the first tooth member is positioned more distally along the axial direction of the cutting assembly compared to the second tooth member. The relative sizes of the retaining features may vary. Further, the shapes of the retaining features may vary. For example, instead of tooth members with sharp ends, the retaining features may be protrusions with round ends, flat ends, etc.

As used herein and in the appended claims, the singular forms “a”, “an”, and “the” include plural referents unless the context clearly dictates otherwise.

The publications discussed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates which may need to be independently confirmed.

It should be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the following description does not explicitly state, in a particular instance, that such combinations or substitutions are possible. It is explicitly acknowledged that express recitation of every possible combination and substitution is overly burdensome, especially given that the of each and every such combination and substitution will be readily recognized by those of ordinary skill in the art.

In many instances, entities are described herein as being coupled to other entities. It should be understood that the terms “coupled” and “connected” (or any of their forms) are used interchangeably herein and, in both cases, are generic to the direct coupling of two entities (without any non-negligible (e.g., parasitic) intervening entities) and the indirect coupling of two entities (with one or more non-negligible intervening entities). Where entities are shown as being directly coupled together, or described as coupled together without description of any intervening entity, it should be understood that those entities can be indirectly coupled together as well unless the context clearly dictates otherwise.

While the embodiments are susceptible to various modifications and alternative forms, specific examples thereof have been shown in the drawings and are herein described in detail. It should be understood, however, that these embodiments are not to be limited to the particular form disclosed, but to the contrary, these embodiments are to cover all modifications, equivalents, and alternatives falling within the spirit of the disclosure. Furthermore, any features, functions, steps, or elements of the embodiments may be recited in or added to the claims, as well as negative limitations that define the inventive scope of the claims by features, functions, steps, or elements that are not within that scope.

What is claimed is:

1. A microsurgical cutting and grasping instrument, comprising:
an actuating assembly including a piston, a cap, and a tension member in operative engagement with the piston and the cap;
an outer shell housing the piston;
a cannula connected to the piston;
a rod axially disposed in the cannula and secured in the outer shell against axial movement;
wherein the cannula is moved in an axial direction by the piston when the outer shell and the piston are squeezed together; and
a cutter assembly disposed at a distal end of the rod and configured to perform both an inline cutting action and a grasping action on a target.

2. The microsurgical cutting and grasping instrument of claim 1, wherein the cutter assembly comprises a first cutting member and a second cutting member separated by a slot, the first cutting member provided with a first cutting edge at the first distal end and the second cutting member provided with a second cutting edge at a second distal end, each of the first and the second cutting edges having one of an arcuate or straight configuration, wherein the first cutting edge and the second cutting edge oppose each other such that when the cannula is moved in the axial direction towards the first and second distal ends of the cutter assembly, the first and second cutting edges engage each other to establish the inline cutting action.

3. The microsurgical cutting and grasping instrument of claim 2, wherein one of the first cutting member and the second cutting member includes a bend along the axial direction.

4. The microsurgical cutting and grasping instrument of claim 2, wherein the first distal end and the second distal end of the cutter assembly are each configured with a recess opposing one another and are movable relative to one another, such that when the cannula is moved in the axial direction towards the first and second distal ends of the cutter assembly, the two opposing recesses establish the grasping action.

5. The microsurgical cutting and grasping instrument of claim 4, wherein the grasping action includes forming the two opposing recesses together into a common recess for retaining a cut portion of the target after the inline cutting action.

6. The microsurgical cutting and grasping instrument of claim 1, wherein the inline cutting action forms a substantially straight cross-section on the target.

7. The microsurgical cutting and grasping instrument of claim 4, wherein the inline cutting action and the grasping action are established with a single movement of the cannula in the axial direction.

8. The microsurgical cutting and grasping instrument of claim 3, wherein one of the first cutting member and the second cutting member includes one or more retaining features.

9. The microsurgical cutting and grasping instrument of claim 8, wherein the one or more retaining features includes sharp end.

10. The microsurgical cutting and grasping instrument of claim 3, wherein one of the first cutting member and the second cutting member includes two or more retaining features having different sizes.

11. The microsurgical cutting and grasping instrument of claim 1, wherein the outer shell is configured to releasably attach to a handle.

12. The microsurgical cutting and grasping instrument of claim 2, wherein one of the first cutting edge and the second cutting edge includes a curved-out portion.

13. The microsurgical cutting and grasping instrument of claim 12, wherein the cured-out portion is shaped as a slot and sized to accommodate the target.

14. The microsurgical cutting and grasping instrument of claim 12, wherein the cured-out portion has an arc shape.

15. The microsurgical cutting and grasping instrument of claim 12, wherein at least 50% of the diameter/lateral dimension of the target is subsumed inside the curved-out portion.
